(19) United States
(12) Patent Application Publication
(10) Pub. No.: US 2008/0260675 A1
(43) Pub. Date: Oct. 23, 2008

(54) Depolymerized Scleroglucan for Regulating and Improving the Moisture Content of the Skin

(75) Inventors: Mike Farwick, Essen (DE); Ursula Maczkiewitz, Essen (DE); Maria Mecking, Bottrop (DE); Georg Schick, Chester, VA (US); Ute Wollenweber, Essen (DE)

Correspondence Address: SCULLY SCOTT MURPHY & PRESSER, PC, 400 GARDEN CITY PLAZA, SUITE 300, GARDEN CITY, NY, 11530, US

(73) Assignee: EVONIK GOLDSCHMIDT GMBH, Essen (DE)

(21) Appl. No.: 12/146,692

(22) Filed: Jun. 26, 2008

Related U.S. Application Data
(63) Continuation of application No. 11/448,953, filed on Jun. 7, 2006.

(30) Foreign Application Priority Data
Jun. 7, 2005 (DE) 102005026061.6

(51) Int. Cl. A61K 8/73; A61K 31/716; A61K 38/02; A61K 38/16; A61K 36/18; A61K 31/7088; A61P 17/00; A61Q 5/00; A61Q 19/00

(52) U.S. Cl. 424/70.13; 514/54; 514/2; 514/12; 424/725; 514/44

(57) ABSTRACT

The use of depolymerized scleroglucan alone, or in combination with one or more active ingredients as a moisturizer, and as anti-inflammatory active ingredient for protecting and for restoring a healthy skin barrier in the field of cosmetic or dermatological skincare is disclosed.

### CROSS REFERENCE TO RELATED APPLICATIONS

The present application is a continuation of copending application Ser. No. 11/448,953 filed on Jun. 7, 2006 which claims benefit of and priority to German Patent Application No. 10 2005 026 061.6, filed Jun. 7, 2005.

### FIELD OF THE INVENTION

The present invention relates to the use of depolymerized scleroglucan alone, or in combination with one or more active ingredients, as moisturizers, and as an anti-inflammatory active ingredient for protecting and for restoring a healthy skin barrier in the field of cosmetic or dermatological skincare.

### BACKGROUND OF THE INVENTION

The horny skin (stratum corneum, SC), which is the outermost layer of the skin, is an important barrier layer and is of particular importance for protection against environmental effects. To retain its smoothness, elasticity and suppleness, the skin requires an optimum of water. These findings were confirmed in basic works, inter alia, by Jacobi, and Schuleit and Szakall (Jacobi, J. Appl. Physiol. 12 (3), 403-7, May 1958; Schneider W & Schuleit H, Arch. Klein. Exp. Dermatol. 193 (5), 434-59, December 1951; Szakall A, Arch. Klein. Exp. Dermatol. 206, 374-9, 1957).

An individual releases several deciliters to several liters of water daily into the environment via the skin. The water located in the skin originates from various sources and, according to more recent findings, is present either as vapor or in liquid form, and also adsorbed to proteins. It is not known how much water the epidermis contains, but it can be assumed that a water content of up to 30% is present in some layers of the stratum corneum.

What can be assumed with certainty is that water is capable of migrating through different skin layers. There are various models for the diffusion of the water through the skin layers, although none has as yet been proven conclusive. Analogous to hydrophobic substances which can penetrate through lipid pores into the horny layer, the water is said to be transported by specific so-called “aqueous pores”. These pores are said to have a diameter of 15-25 Å.

Another approach postulates that water-filled channels pass through the stratum corneum. Diffraction experiments with X-rays have been able to show that there are holes in a lipid double-layer system which are large enough in order to be able to collect condensed water therein.

Thus, for regulating moisture in the skin, besides an intact permeability barrier, the presence of water-binding substances which are formed in the epidermal horny layers is undoubtedly decisively required. These natural moisturizing factors (NMFs) contained in the epidermis bind moisture in the skin. The NMFs represent a mixture of different compounds and consist of 40% amino acids, 12% pyrrolidonecarboxylic acid, 7% urea and 41% inorganic and organic salts (primarily lactates).

Drastic environmental conditions, such as, for example, low temperatures or too little moisture in the winter, contribute to a considerable degree to the skin becoming rough and dry. The moisturizing factors present in the epidermis are, in addition, readily removed by frequent washing or bathing. Thus, more water can escape from deeper skin layers and the so-called transepidermal water loss (TEWL) increases, which brings about drying of the skin. It is assumed that the loss of natural moisturizing factors correlates with a reduction in the water content and reduced softness of the keratin layer.

Sensorily, this manifests itself by symptoms, for example, a more rough, flaky, lackluster and dull-looking skin surface. Loss of flexibility and impairment of the barrier function of the skin, which depends on the water-binding capacity of the stratum corneum, are the result. Consequently, the water content of the horny layer is further reduced.

Careful care for preventing constantly dry skin is not only an esthetic requirement, but also a tried and tested means of effectively preventing chronic skin diseases. Here, moisture regulation of the skin can be effectively assisted through topical application of corresponding formulations.

A large number of in vivo methods for determining the moisture content of the skin are known. Here, physical parameters, such as the conductivity and the dielectric properties (capacity) of the horny layer are determined, which correlate directly with the skin moisture. Various instruments are available for determining the hydration of the stratum corneum, such as, for example, the corneometer models CM 820 and CM 825 (Courage+Khazaka), and the “dermal phase meter” Skicon 200 (Nova). These noninvasive and simple methods allow a change in the skin moisture to be measured quantitatively. Moreover, the viscoelasticity of the skin can be determined using the Dermal Torque Meter (DiaStron) or by using the Cutometer (Courage+Khazaka).

In order to counteract a dry skin condition and to restore the water balance in the skin, there are a number of cosmetic preparations with a hydroregulative effect. These preparations are, in the form of emulsions, ideal preparations for supplying fat and moisture to the skin and generally comprise a number of active ingredients which develop a protective function upon application, thereby improving the condition of the skin surface and changing the functional state of the skin by, for example, having a regulating effect on the skin moisture, and care properties taking effect as a result of penetration under the surface of the skin.

There are various mechanisms for positively influencing the epidermal water content through cosmetic ingredients and formulations. For example, the evaporation of water from the upper layers of the skin can be suppressed by an occlusive lipid or polymer film. As a result, water is released from the lower skin layers to the upper skin layers and the formation of perspiration is reduced, as a result of which the skin moisture of the upper layers of the SC increases considerably. Under such occlusive conditions, however, this typically results in a build-up of water in the skin and increased endogenous swelling of the horny layer, as a result of which the ability of the skin to regenerate is slowed.

From a formulation point of view, it is possible to produce cosmetic products which comprise more water than the stratum corneum and thus, upon penetration of the intact formulation, release water to the SC. Special lipids are likewise able to reduce the transepidermal water loss and can therefore also be regarded as being a type of moisturizer.

A further customary approach is the addition of moisturizers as activating ingredients to cosmetic emulsions which are intended to ensure that the keratin layer is supplied with an adequate amount of moisture over defined time intervals. Moisturizers are also referred to as humectants and are intended, on the one hand, to retain water in the epidermis, on the other hand to reduce the TEWL by stabilizing the barrier function in the upper horny layer.

A large number of such substances has been described and is already used. These generally have the ability to bind water to a greater or lesser degree and to completely or partially replace the washed-out natural substances. In principle, these include hygroscopic substances such as, in particular, polyhydric alcohols, ethoxylated polyols, sugars, and polysaccharides, such as, for example, the skin's own moisturizer hyaluronic acid and its salts, which holds an important role for the regulation of moisture since it can bind water in the stratum corneum. This ultimately results in an improvement in the skin elasticity.

Scleroglucan (INCI: Sclerotium Gum, chemically: β-1,3-glucan) is a polysaccharide of microbiological origin. Every third glucose unit is joined to a further glucose via a β-1,6 bond (general formula). In the native form, scleroglucan forms, in neutral medium, the triple helix typical of β-glucan having a molecular weight of 3-5×10⁶. The maximum solubility in water at a neutral to weakly acidic pH is about 1.5-2%.

The general formula of scleroglucan is as follows:

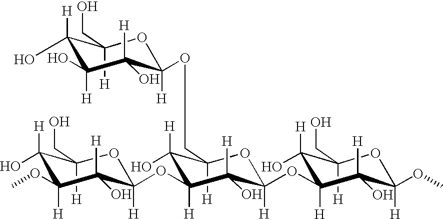

The highly thickening properties of scleroglucan are widely known and described in various patents (see, for example, EP 0 979 642, WO 00/60198, and JP 612 675/03).

Furthermore, on account of the antiviral and antibacterial effect of β-glucans, uses in medical and food sector of this biopolymer class have been described, e.g., WO 98/04082, and GB 2 050 825. It is generally known that β-glucans exhibit immunostimulating effects, for which reason this class of substances are attractive active ingredients for antiaging and sun care applications (WO 98/04082).

Besides the thickening property, the improvement in skin hydration following topical application of a cosmetic formulation comprising scleroglucan was presented in U.S. Pat. No. 6,162,449. The described scleroglucan has an average molecular weight of 1-12×10⁶. Besides skin moisturization, the anti-inflammatory effect and also the pleasant feel on the skin of the end formulation are also highlighted here. The product form is a 1% strength solution, which can be added to the formulation in an amount up to 10%.

### SUMMARY OF THE INVENTION

Surprisingly, it has now been found that depolymerized scleroglucan has very much greater effectiveness in the area of skin hydration compared with the high molecular weight derivative.

Moreover, the depolymerized scleroglucan can ultimately be incorporated into the end formulation in relatively high concentrations because of its lower molecular weight and the associated lower viscosity.

In addition, it has surprisingly been found that depolymerized scleroglucan has anti-inflammatory activity and is thus suitable for applications in anti-inflammatory and aftersun skincare preparations.

Accordingly, the present invention presents a β-1,3-scleroglucan of average molecular weight from 8×10² to 1×10⁶ preferably 1×10⁵ to 7×10⁵, which can be incorporated into the end formulation with a suitable carrier in an end concentration of from 0.05 to 6.0, preferably 0.2 to 2.0% by weight.

The cosmetic composition corresponding to the present invention may be a skincare formulation, such as, for example, an emulsion or a cream, in which the scleroglucan has one or more actions, such as, for example, an improvement in the moisture content, and also film-forming and thus protective properties on the skin. The film-forming effect also gives the skin a soft and silky feel.

The skincare formulation can be formulated as an aqueous lotion, a water-in-oil or oil-in-water emulsion, an oil or oil-alcohol lotion, a vesicular dispersion of anionic or nonionic amphophilic lipids, an aqueous, water-alcohol, an alcohol or oil-alcohol gel, a solid stick or as an aerosol.

If the inventive skin-care formulation is formulated as a water-in-oil or oil-in-water emulsion, the cosmetically acceptable carrier preferably comprises 5 to 50% of an oil phase and 47 to 94.95% water, based on the total amount of the formulation.

The oil phase can comprise any cosmetic oil or a mixture thereof Examples of such oils include aliphatic hydrocarbons, such as liquid paraffin, squalane, Vaseline and ceresine, vegetable oils, such as olive oil, almond oil, sesame oil, avocado oil, castor oil, cocoa butter and palm oil, animal oils, such as shark liver oil, cod liver oil, whale oil, beef tallow, butterfat, waxes, such as beeswax, carnauba palm wax, spermaceti and lanolin, fatty acids, such as lauric acid, myristic acid, palmitic acid, stearic acid, oleic acid and behenic acid; aliphatic alcohols, such as lauryl, stearyl, cetyl and oleyl alcohol and aliphatic esters, such as isopropyl, isocetyl or octadecyl myristate, butyl stearate, hexyl laurate, diisopropyl ester of adipic acid or diisopropyl sebacate. Preferred mono- or polyols for use in oil-alcohol lotions or oil-alcohol or alcohol gel include ethanol, isopropanol, propylene glycol, hexylene glycol, glycerol and sorbitol.

Cosmetic formulations corresponding to the present invention have a moisturizing and skin-calming effect.

Besides the formulation constituents customary in the cosmetic and dermatological field, it is also possible to co-use biogenic active ingredients or active ingredient combinations. Biogenic active ingredients are understood as meaning, for example, tocopherol, tocopherol acetate, tocopherol palmitate, ascorbic acid, deoxyribonucleic acid, retinol, bisabolol, allantoin, phytantriol, panthenol, α-hydroxy acids, amino acids, hyaluronic acid, creatine (and creatine derivatives), guanidine (and guanidine derivatives), ceramides, phytosphingosine (and phytosphingosine derivatives), sphingosine (and sphingosine derivatives), pseudoceramides, essential oils, peptides, protein hydrolysates, plant extracts and vitamin complexes.

Besides skincare products of the composition described above, they may also be hair care formulations, such as shampoos and/or conditioners, in which the scleroglucan is incorporated and exerts a calming effect on a scalp irritated as a result of chemical treatment.

### BRIEF DESCRIPTION OF THE DRAWINGS

FIG. 1 is a graph showing the water-retention capacity of various formulations.

FIG. 2 is a graph showing the increase in relative corneometry units of various formulations.

FIG. 3 is a graph comparing the anti-inflammatory effect of the inventive formulation as compared to native scleroglucan.

FIG. 4 is a graph illustrating the LDH release 24 hours following application of various formulations to the skin.

### DETAILED DESCRIPTION OF THE INVENTION

As stated above, the present invention relates to depolymerized scleroglucan and its use for regulating and improving the moisture content on the skin. More particularly, the present invention relates to a β-1,3-scleroglucan having an average molecular weight from 8×10² to 1×10⁶, preferably 1×10⁵ to 7×10⁵, which can be incorporated into an end formulation with a suitable carrier in an end concentration of from 0.05 to 6.0, preferably 0.2 to 2.0% by weight.

The depolymerized scleroglucan employed in the present application has the formula:

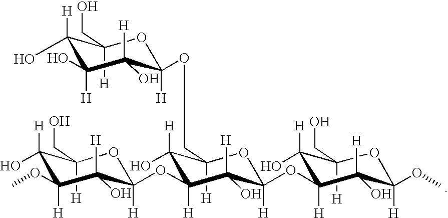

High molecular weight scleroglucan with a molecular weight of from 1×10⁶ to 12×10⁶ is produced by fermentation of Sclerotium rolfsii ATCC 15205. For this, the fungus Sclerotium rolfsii ATCC 15205 is cultivated in a culture medium under microaerobic conditions. The medium here contains a carbon source, preferably glucose, a nitrogen source, a phosphorus source, potassium chloride, magnesium sulfate heptahydrate, iron(II) sulfate heptahydrate, and yeast extract.

Finally, the high molecular weight scleroglucan is isolated by ending the cultivation process by agitation at a temperature of 15°-40° C. and separating the cell mass from the liquid. The high molecular weight scleroglucan can, for depolymerization, be squeezed as a solution through a capillary so that the polysaccharide chains are broken down due to shear forces. Suitable solvents for this process are water, acetone or benzene mixed with organic solvents such as methanol, ethanol, isopropanol or n-propanol and tetrahydrofuran. The degree of depolymerization is dependent on various physical parameters such as pressure, and also diameter and length of the capillary. A defined degree of depolymerization is achieved by repeating the procedure.

Alternatively, depolymerization of scleroglucan can, for example, be achieved by incubating the high molecular weight derivative together with an aqueous solution of sodium hydroxide and hydrogen peroxide.

After the described treatment, the depolymerized scleroglucan has the three-dimensional structure of a triple helix consisting of β-1,3-bonded glucopyranose as a main chain and β-1,6-bonded glucopyranose as a side chain with an average molecular weight of 8×10² to 1×10⁶, preferably 1×10⁵ to 7×10⁵, particularly preferably 2×10⁵ to 6×10⁵. The three-dimensional structure of the depolymerized β-1,3-scleroglucan can be determined experimentally, for example by means of light-diffraction technology methods or gel filtration.

### FORMULATION EXAMPLES

The formulations according to the invention are prepared in the usual manner; the depolymerized scleroglucan is preferably dissolved in the aqueous phase of the formulation. Typical guide formulations for skin-treatment compositions belong to the prior art and are contained, for example, in the brochures of the manufacturers of the respective basic and active ingredients. Scleroglucans can, in general, be present in a concentration of from 0.05 to 6.0% by weight.

A typical emulsion (W/O or O/W) comprises, for example:
a. 0.05% to 6.0% by weight of depolymerized scleroglucan,
b. 0 to 10% by weight of one or more emulsifiers,
c. 0 to 10% by weight of one or more consistency regulators,
d. 0 to 30% by weight of one or more cosmetic oils or emollients
   and customary auxiliaries and additives in customary concentrations.

The following examples are provided to demonstration the effectiveness of the depolymerized scleroglucans of the present invention

In order to avoid possible effects of the ingredients present in cosmetic formulations, for carrying out the demonstrations of effectiveness, a standard formulation was prepared which is summarized in Table 1. Furthermore, in order to investigate the effect of the degree of polymerization of the scleroglucan proposed in the present invention, products of various chain length were used (see in this regard Table 2).

TABLE 1
O/W cream

| | |
|---|---|
| Ceteareth-25 | 2.0% |
| Glyceryl stearate | 4.0% |
| Stearyl alcohol | 2.0% |
| Ethylhexyl stearate | 8.5% |
| Caprylic/capric triglyceride | 8.5% |
| Preservative | 0.1% |
| Water | ad 100.0% |
| Scleroglucan | x % |

TABLE 2
Depolymerized scleroglucan of various molecular weights

| | MW* |
|---|---|
| Native scleroglucan | 2 × 10⁶ |
| Depolymerized product 1 | 520,000 |
| Depolymerized product 2 | 500,000 |
| Depolymerized product 3 | 400,000 |
| Depolymerized product 4 | 375,000 |

*MW = molecular weight

The examples below are intended to illustrate the subject-matter of the invention in more detail.

### EXAMPLE 1

### Determination of the Water-Retention Capacity Ny Means of an Artificial Carrier Material

The water-retention capacity was determined using a carrier material (Vitro Skin™ Substrate, IMS Incorp., Milford, USA), onto which, following evaluation of the net weight, the cosmetic formulation to be tested was applied and weighed once more.

The formulation (Table 1, comprising 2% of the products 1-4 or the native scleroglucan) was applied by dabbing the substrate with a spatula and then spreading. Afterwards, the samples were incubated for 4 hours at 22° C. and 72% relative humidity and, finally, weighed again; the initial and final weighing took place in a climatically controlled chamber under defined conditions (22° C., 55% relative humidity). The relative weight (=water) loss was calculated.

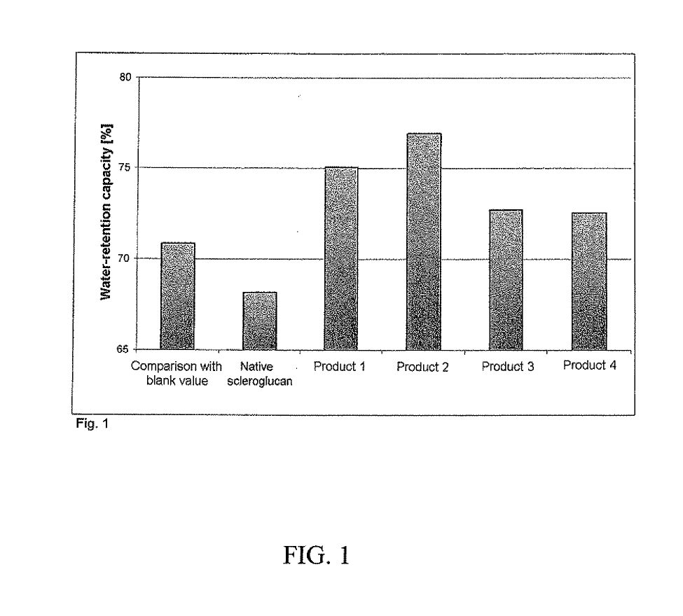

The average values shown in FIG. 1 consist of at least 15 individual determinations.

Specifically, FIG. 1 illustrates the effective water-retention capacity of depolymerized scleroglucan, in particular of product 2, compared with a control formulation (“comparison with blank value”), and also the polymeric starting material.

### EXAMPLE 2

### Determination of the Skin Moisture In vivo By Means of Corneometry

Corneometry is a noninvasive method which, on the basis of changes in physical parameters within the stratum corneum, permits conclusions with regard to skin moisture. In practice, 12 to 15 subjects were recruited whose skin moisture was measured before and after a single application of the test formulations using a Corneometer HM99 (Courage & Khazaka, Cologne, Germany) in accordance with the manufacturer's instructions.

Following climatization of the subjects for 15 minutes under defined conditions of 22° C. and 55% relative humidity, the skin hydration was determined corneometrically and, subsequently thereto, 50 mg of the test formulation (Table 1, comprising 1% of product 1-4, and the native starting material) were applied to an area of skin each with a diameter of 2.5 cm, and after two minutes any residues of the formulation were removed.

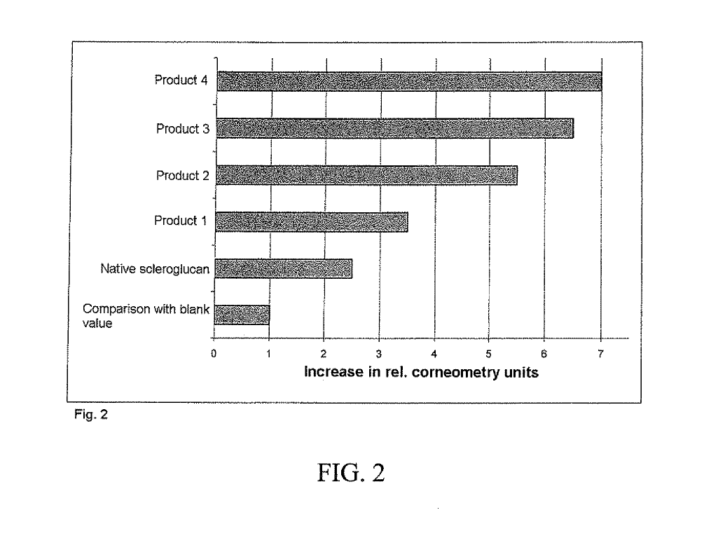

After a contact time of two hours, the skin hydration was measured again following climatization of 15 minutes (22° C., 55% rel. humidity). An increase in the relative corneometer units CU correlates directly with improved skin hydration. This result is shown, for example, in FIG. 2.

Specifically, FIG. 2 shows clearly that depolymerized scleroglucan, specifically product 4, compared with the native starting material, was much more effective for skin hydration.

### EXAMPLE 3

### Determination of the Anti-Inflammatory Effectiveness By Means of a Curative Study

In a curative study carried out at the Dr. Schrader Institute (Holzminden, Germany), the anti-inflammatory effectiveness of the depolymerized scleroglucan product 4 compared with the polymeric starting material was demonstrated. For this, 20 subjects were recruited, who apply the test formulations (see Table 1) comprising 0.5% active ingredient to the areas of skin that were later irradiated over a period of 3 days before the start of and during the test. The test area used was the back. Firstly, the MEDu (minimal erythemal dose of the untreated skin) of the subjects was determined and on the next day the starting situation was then documented by means of a chromameter and a spectral photometer.

Then (=day 0), the skin was irradiated with UV light using the sun simulator SU 5000 according to Schrader corresponding to the COLIPA irradiation spectrum. The radiation dose was fixed at 1.75 times the MEDu and brought about a slight erythema reaction of the skin. Following irradiation, the subjects were instructed to apply the test formulations to the areas for which they were intended twice daily.

In parallel to the daily product application, in each case remission spectroscopic and color measurements were carried out in the Institute. Thus, skin reddening before and also 24 and 48 hours after irradiation was ascertained quantitatively in the form of remission absorption integrals.

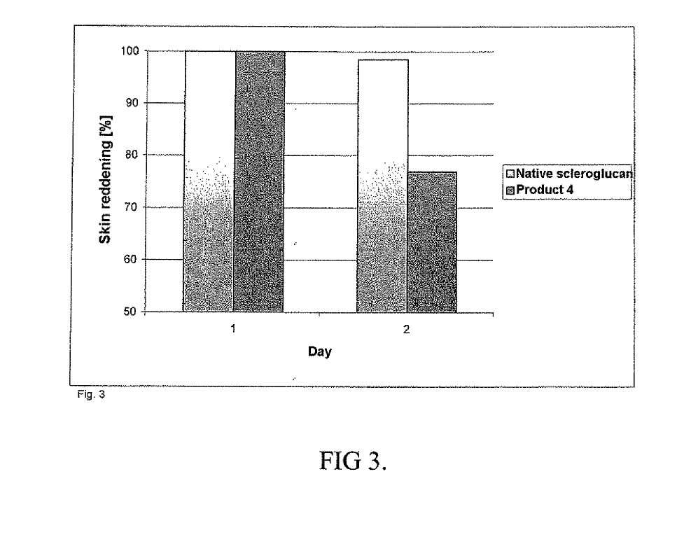

FIG. 3 shows the skin reddening in % on day 1 (=24 hours after irradiation), on which the erythema reaches its maximum, and also on day 2. According to this, the depolymerized scleroglucan product 4 showed an anti-inflammatory effect compared with the native starting material since the skin reddening subsides noticeably more quickly.

### EXAMPLE 4

### Determination of the Anti-Inflammatory Effectiveness on an Artificial Skin Model

In an in vitro study on the artificial skin model SkinEthic™ (SkinEthic Laboratories, Nice, France), the anti-inflammatory effectiveness of the depolymerized scleroglucan product 4 was investigated.

For this, at the start of the test series, in each case 30 μl of either 0.9% sodium chloride solution (“comparison with blank value”) or of product 4 in a concentration of 0.1 mg/ml or 1 mg/ml were applied to the artificial skin for 24 hours.

In order to cause an inflammation reaction on the skin model, 30 μl of a 0.25% aqueous sodium dodecyl sulfate solution were then applied and were only washed off after 40 minutes with medium for cultivating the artificial skin. Sodium dodecyl sulfate (SDS) is known as a detergent with a high degree of skin irritancy. Following the damage, again in each case 30 μl of either 0.9% sodium chloride solution (“comparison with blank value”) or of product 4 in a concentration of 0.1 mg/ml or 1 mg/ml were applied to the artificial skin.

As a parameter of the cell damage, the enzyme lactate dehydrogenase (LDH) was determined; this was released in damaged cells from the cytosol and could thus be detected photometrically in the cell supernatant. Cell damage was determined directly following the SDS application and also 24 hours afterwards.

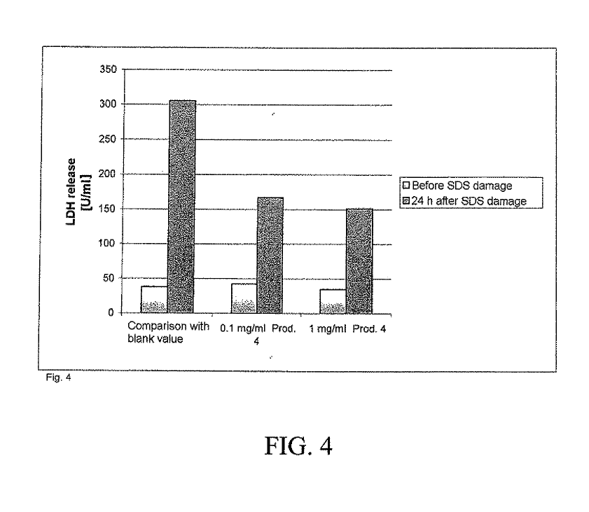

FIG. 4 shows the LDH release 24 hours following application to the skin model.

According to this, the depolymerized scleroglucan product 4 had a significant anti-inflammatory effectiveness even in a concentration of 0.1 mg/ml since the LDH release could be reduced to approximately half.

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A method of protecting and/or restoring a healthy skin barrier comprising applying a cosmetic composition to skin, wherein said composition comprises, as an active ingredient, 0.05 to 6.0% by weight of a β-1,3-scleroglucan of the general formula 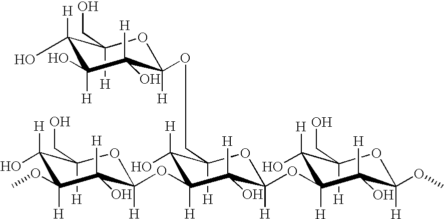
and having an average molecular weight of from 2×10⁵ to 6×10⁵, and a skincare or hair care acceptable carrier for said active ingredient.

2. The method according to claim 1 wherein the cosmetic composition acts as a moisturizer to the skin.

3. The method according to claim 1 wherein the cosmetic composition acts as an anti-inflammatory agent to the skin.

4. The method according to claim 1 wherein the cosmetic composition is used as an aftersun skincare agent.

5. The method according to claim 1 wherein the cosmetic composition is used as a hair care agent.

6. The method according to claim 1 wherein the composition further comprises 0 to 10% by weight of one or more emulsifiers, 0 to 10% by weight of one or more consistency regulators, 0 to 30% by weight of one or more surfactants, 0 to 30% by weight of one or more cosmetic oils or emollients, and usual auxiliaries and additives.

7. The method according to claim 1 wherein the composition further comprises at least one compound selected from the group consisting of tocopherol and derivatives, ascorbic acid and derivatives, deoxyribonucleic acid, retinol and derivatives, alpha-lipoic acid, niacinamide, ubiquinone, bisabolol, allantoin, phytantriol, panthenol, α-hydroxy acids, amino acids, hyaluronic acid, polyglutamic acid, creatine and creatine derivatives, guanidine and guanidine derivatives, ceramides, sphingolipids, phytosphingosine and phytosphingosine derivatives, sphingosine and sphingosine derivatives, sphinganine and sphinganine derivatives, pseudoceramides, essential oils, peptides, proteins, protein hydrolysates, plant extracts and vitamin complexes.

8. The method according to claim 1 wherein the β-1,3-scleroglucan of the composition is present in a concentration from 0.2 to 2% by weight.
